(19) United States
(12) Patent Application Publication
(10) Pub. No.: US 2004/0082048 A1
(43) Pub. Date: Apr. 29, 2004

(54) Genes and protein sequences useful as drug targets for therapeutic action against protozoa

(76) Inventors: Nittala Venkata Narasimha Viswanath, Hyderabad (IN); Jagannadham Kodam, Secunderabad (IN)

Correspondence Address: VENABLE, BAETJER, HOWARD AND CIVILETTI, LLP, P.O. BOX 34385, WASHINGTON, DC, 20043-9998, US

(21) Appl. No.: 10/270,311

(22) Filed: Oct. 15, 2002

(51) Int. Cl. C12N 9/99; C12N 9/02; C12N 9/12; C12N 9/24; C12N 1/10; C07H 21/04

(52) U.S. Cl. 435/184; 435/189; 435/194; 435/200; 536/23.7; 435/258.2

(57) ABSTRACT

Novel gene and protein sequences useful as drug targets for therapeutic action include at least one enzyme selected from the group consisting of (a) aldolase, (b) lactate dehydrogenase, (c) 3-phosphoglycerate kinase, (d) carbamoyl phosphate synthase, (e) glutamine amido transferase, (f) chitinase, (g) amino acyl synthetase, and (h) trypsin inhibitor (Kunitz protease inhibitor).

### FIELD OF INVENTION

This invention relates to a novel genes and protein sequences of plasmodium Falciparum which can be potential drug targets for therapeutic action against the protozoa.

### BACKGROUND OF THE INVENTION

Malaria is a devastating parasitic disease transmitted through the bite of infected Anopheles mosquitoes. The most common of four human malaria species are Plasmodium Falciparum, Malariae, Ovale, and Vivax. By contrast, P. Falciparum is the most deadly species and thus forms the subject matter of most malaria-related research.

There are several factors that make the development of malaria vaccine very difficult. Firstly, the size and secondly, the genetic complexity of the parasite. As each infection presents thousands of antigens into the human immune system, it becomes very difficult to ascertain the useful target for vaccine development and to date at least 40 promising antigens have been identified. The malarial parasite changes several stages of life in the human host, presenting a different subset of molecules for the immune system to combat at each stage. The parasite has evolved a series of strategies and misdirects human immune system.

An ideal drug target is generally an enzyme/receptor in a pathway and its inhibition leads to either killing a pathogenic organism or to modify some aspects of metabolism of body that is functioning normally. An ideal target should have the following characteristics:

1. Is essential for the survival of the organism.

2. Located at a critical step in the metabolic pathway.

3. Makes the organism vulnerable.

4. The enzyme amendable for simple High Throughput Screening (HTS) assays.

P. Falciparum has a very complicated life-cycle. Up untilnow, therapeutic intervention has been based on traditional medicines or their derivatives e.g quinine, paraquinine, chloroquinine etc. Many targets were reported for therapeutic action against malaria.

The parasite infects humans and not the mosquito. This is an interesting aspect of the disease mechanism. Identification of the metabolites in the parasite that interfere with human metabolites is a possible way of understanding the disease mechanism. Identification of a unique target for the parasite is akin to find the unique genes in the Malaria causing parasite, Plasmodium Falciparum. Novel drug target identification work was undertaken by using bioinformatics tools and databases.

U.S. Pat. No. 6,066,623 relates to a method of controlling malaria in mammals by injecting a polynucleotide delivering vector into a mammal, wherein said vector comprises atleast one DNA sequence encoding a plasmodium species proteins operably linked to a mammalian specific protein.

U.S. Pat. No. 5,597,708 relates to the cloning of gene P.195 of P.Falciparum and also relates to a vaccine comprising the P.195 protein and to the use of the same in the prophylaxis of malaria.

U.S. Pat. No. 5,585,268 and No. 5,573,943 relates to antigen 41 kD and 42 kDa of the P. Falciparum.

Further, U.S. Pat. No. 5,565,327 relates to CTD kinase of sporozoan parasites displays a specificity distinct from the analogous activity in mammalian cells and U.S. Pat. No. 6,242,428 relates to novel nucleosides and nucleoside dimers containing an L-sugar.

Thus, existing patents relate to individual enzymes as targets, whereas the approach of the present invention is to target these enzymes (Aldolase, Lactate dehydrogenase, 3-Phosphoglycerate kinase) as a unit as the glycolysis pathway is unique for malarial parasite. The present invention will help to manufacture a drug which will be capable of attracting the three enzymes. P. Falciparum develops drug resistance and these attract the three enzymes as a unit will provide better results in the treatment of malaria.

### OBJECTS OF THE INVENTION

An object of this invention is to identify novel genes and protein sequences of Plasmodium Falciparum.

A further object of this invention is to propose a gene can be a potential drug target for therapeutic action.

A still further object of this invention is to propose the genes protein product which plays a crucial role for its survival and replication can be targeted to facilitate the prevention of malaria.

### SUMMARY OF THE INVENTION

This invention relates to a novel gene or protein sequences which are possible drug targets for therapeutic action which comprises the enzymes/Proteins (a) Aldolase, (b) Lactate dehydrogenase, (c) 3-Phosphoglycerate kinase, (d) Carbamoyl phosphate synthase, (e) Glutamine amidotransferase, (f) Chitinase (g) Amino acyl synthetase (h) Trypsin inhibitor (Kunitz protease inhibitor).

### DETAILED DESCRIPTION OF THE INVENTION

According to this invention there are novel genes and protein sequences of Plasmodium Falciparum. For identification of these genes and protein sequences of P. Falciparum were submitted to online Basic Local Alignment and Search Technique (BLAST) at National Center Biotechnology Information (NCBI) site in an automated manner. The molecular databases, which were used for comparing sequences, are Human EST, Human, cDNA, Non-redundant, Swiss-Prot, dbEST and Microbial Genomes. BLAST results were analyzed to identify unique gene sequences based on the statistical score. 4∅ sequences were found unique to P. Falciparum. These included genes and protein sequences, some had annotation information and others were not annotated. Annotation of all the proteins indicated following enzymes/proteins as crucial for survival and replication of Plasmodium Falciparum. The unique sequences identified are:

(a) Aldolase,

(b) Lactate dehydrogenase

(c) 3-Phosphoglycerate kinase

(d) Carbamoyl phosphate synthase

(e) Glutamine amidotransferase

(f) Chitinase

(g) Amino acyl synthetase

(h) Trypsin inhibitor (Kunitz protease inhibitor)

In the above list (a)-(c) are active in the Glycolysis pathway of P. Falciparum. This pathway is different compared to all species.

The details of the enzyme are:

Description: Fructose-bisphosphate aldolase (EC 4.1.2.13) is a glycolytic enzyme that catalyzes the reversible aldol cleavage or condensation of fructose-1,6-bisphosphate into dihydroxyacetone-phosphate and glyceraldehyde 3-phosphate.

D-fructose 1,6-bisphosphate<=>glycerone phosphate+D-glyceraldehyde 3-phosphate.

(b) Lactate Dehydrogenase

EC ID: 1.1.1.27

Description: L-lactate dehydrogenase (EC 1.1.1.27) (LDH) catalyzes the reversible NAD-dependent interconversion of pyruvate to L-lactate.

(s)-lactate+NAD(+)<=>pyruvate+NADH

(c) Phosphoglycerate Kinase

EC ID: 2.7.2.3

Description: Phosphoglycerate kinase (EC 2.7.2.3) (PGK) catalyzes the second step in the second phase of glycolysis, the reversible conversion of 1,3-diphospho-glycerate to 3-phosphoglycerate with generation of one molecular of ATP.

ATP+3-phospho-D-glycerate<=>ADP+3-phospho-D-glyceroyl phosphate

By the present invention it is possible to design drug leads, which attack the three enzymes as a unit rather than individual enzymes. As P. Falciparum develops drug resistance, attacking the three as unit will provide improved results than a single enzyme or target.

Description about rest of the targets in given below.

(d) Carbamoyl Phosphate Synthase

EC ID: 6.3.4.16

Description: Carbamoyl-phosphate synthase (CPSase) catalyzes the ATP-dependent synthesis of carbamyl-phosphate from glutamine (EC 6.3.5.5) or ammonia (EC 6.3.4.16) and bicarbonate. This important enzyme initiates both the urea cycle and the biosynthesis of arginine and pyrimidines.

2ATP+NH₃+CO₂+H₂O <=>2ADP+phosphate+carbamoyl phosphate

Blocking this enzyme can effectively stop the replication of P. Falciparum, thus this enzyme can be important target.

(e) Glutamine Amidotransferase

EC ID: 2.4.2.14

Description: Amido phosphoribosyl transferase is involved in Purine biosynthesis.

5-phospho-beta-D-ribosylamine+diphosphate+L-glutamate<=>L-glutamine+5-phospho-alpha-D-ribose 1-diphosphate+H(2)O

This enzyme is not yet indexed in P. Flaciparum metabolic pathways database. This is the result of annotating a hypothetical protein (gi 8248745) in the NCBI database. Blocking of this enzyme will effectively stop the replication of P. Falciparum. Hence this enzyme will be an important drug target as mentioned in the claims.

(f) Chitinase

EC ID: 3.2.1.14

Description: Chitinases are enzymes that catalyze the hydrolysis of the beta-1,4-N-acetyl-D-glucosamine linkages in chitin polymers. O-Glycosyl hydrolases (EC 3.2.1−) are a widespread group of enzymes which hydrolyse the glycosidic bond between two or more carbohydrates or between a carbohydrate and a non-carbohydrate moiety.

This enzyme is not yet indexed in P. Falciparum metabolic pathways database. This is the result of annotating a hypothetical protein (gi 7494226) in the NCBI database. Blocking of this enzyme will effectively stop the replication of P. Falciparum. Hence this enzyme will be an important drug target as mentioned in the claims.

(g) Aminoacyl-Transfer RNA Synthetases

EC ID:6.1.1.

Description: Aminoacyl-tRNA synthetases (EC 6.1.1.−) are a group of enzymes which activate amino acids and transfer them to specific tRNA molecules as the first step in protein biosynthesis. In prokaryotic organisms there are at least twenty different types of aminoacyl-tRNA synthetases, one for each different amino acid.

This enzyme is not yet indexed in P. Falciparum metabolic pathways database. This is the result of annotating a hypothetical protein (gi 7494218) in the NCBI database. Blocking of this enzyme will effectively stop the replication of P. Falciparum. Hence this enzyme will be an important drug target as mentioned in the claims.

(h) Trypsin Inhibitor (Kunitz Protease Inhibitor)

This is not an enzyme, but is an important surface membrane protein. The sequence has similarity to cytoadherence in P. Falciparum. The soyabean trypsin inhibitor (Kunitz) family (STI) is one of the numerous families of proteinase inhibitors. It comprise plant proteins which have inhibitory activity against serine proteinases from the trypsin and subtilisin families, thiol proteinases and aspartic proteinases as well as some proteins that are probably involved in seed storage. The STIs belong to a super family that also contains the interleukin-1 proteins, heparin binding growth factors (HBGF) and histactophilin, all of which have very similar structures, but share no sequence similarity with the STI family.

Inhibitors from cereals are active against subtilisin and endogenous alpha-amylases, while some also inhibit tissue plasminogen activator. The inhibitors are usually specific for either trypsin or chymotrypsin, and some are effective against both. They are thought to protect the seeds against consumption by animal predators, while at the same time existing as seed storage proteins themselves—all the activity inhibitory members contain 2 disulphide bridges. The existence of a member with no inhibitory activity, winged bean albumin 1, suggests that the inhibitors may have evolved from seed storage proteins.

This hypothetical protein has vague resemblance to PolyA+RNA, from asynchronous blood stage parasites of the Dd2 isolate cultured in vitro. This indicates that the protein in P. Falciparum acts as an inhibitor against attacks from immune system.

This protein is not yet indexed in P. Falciparum database. This is the result of annotating a hypothetical protein (gi 3845319) in the NCBI database. Blocking of this protein will effectively stop the replication of P. Falciparum. Hence this protein will be an important drug target as mentioned in the claims.

We claim:

1. A novel gene and protein sequences as drug targets for therapeutic action which comprises the enzymes (a) Aldolase, (b) Lactate dehydrogenase, (c) 3-Phosphoglycerate kinase, (d) Carbamoyl phosphate synthase (e) Glutamine amidotransferase, (f) Chitinase, (g) Amino acyl synthetase (h) Trypsin inhibitor (Kunitz protease inhibitor).

2. The novel gene and protein sequences as claimed in claim 1 is identified by prefering [symbol] sequence comparison of gene and protein sequences against avilable molecular biology sequence databases.

3. The novel gene or protein sequences as claimed in claim 1 wherein enzymes (a), (b) & (c) are unique to glycolysis pathway and together form targets for P. Falciparum.

4. The novel gene or protein sequences as claimed in claim 1 wherein the enzyme (d) to (h) individually form possible drug targets for P. Falciparum.

5. The novel gene or protein sequences as claimed in claim 1 are unique to P. Falciparum.

6. The novel gene and protein sequences as claimed in claim 1 wherein the replication of P. Falciparum in the host human cell can be inhibited when the activity of the said enzymes are blocked.
